(19) United States
(12) Patent Application Publication
(10) Pub. No.: US 2016/0139067 A1
(43) Pub. Date: May 19, 2016

(54) BUILDING INTERIOR MONITORING SYSTEMS AND METHODS FOR MONITORING CLEANING AND THE LIKE

(71) Applicant: Mark GRACE, Alpharetta, GA (US)

(72) Inventor: Mark GRACE, Alpharetta, GA (US)

(73) Assignee: EMEH, INC., Lebanon, NJ (US)

(21) Appl. No.: 14/542,998

(22) Filed: Nov. 17, 2014

(51) Int. Cl. G01N 25/00; B08B 13/00; A61L 2/28

(52) U.S. Cl. CPC G01N 25/00; A61L 2/28; B08B 13/00

(57) ABSTRACT

An interior monitoring method, implemented by a server coupled to a plurality of sensors deployed throughout a location, includes receiving data from the plurality of sensors, wherein the plurality of sensors are configured to monitor environmental conditions and provide data based thereon which can be used to determine cleaning and effectiveness of the cleaning for the location; correlating the data; and determining whether the location has been cleaned based on the correlated data. The building interior monitoring systems and methods include deploying tens to hundreds to thousands of sensors per location and continually monitoring data to determine whether the location has been cleaned or whether the location has been damaged, based on the temperature and/or pressure readings. This is particularly advantageous in environments such as hospital rooms, bathrooms, restaurants, locker rooms, etc., i.e., any environment where there is a risk for the spread of disease.

### FIELD OF THE DISCLOSURE

The present disclosure relates generally to building systems and methods. More particularly, the present disclosure relates to building interior monitoring systems and methods for monitoring cleaning, environmental protection, and the like.

### BACKGROUND OF THE DISCLOSURE

In hospitals, restaurants, restrooms, fitness facilities, retailers, and the like, interior components of buildings, such as walls, floors, ceilings, casework furniture, doors, privacy curtains, and the like, have historically been kept clean, safe and refreshed by having a crew routinely and periodically visit the area and clean the interior components. Conventionally, the crew signs a sheet of paper or electronically scans a badge to prove they cleaned the area, and this record is posted to the public to give the public confident that the area is clean and free of disease. Disadvantageously, this approach does not actually measure the degree of cleanliness and effectiveness on the surfaces of the interior components or whether they were actually cleaned and when and how often. Staph infection is growing in hospitals and other public places as a result of contamination of interior components that are not properly cleaned and/or protected from physical abuse and then subsequent contamination. For example, a study published in Infection Control and Hospital Epidemiology found that a high percentage of hospital curtains were contaminated with MRSA, VRE and C-Diff. With close to 18,000 deaths in the United States alone due infections in the hospital, it is clear that proper and effective cleaning to reduce the risk of infection is essential.

A better approach is required to ensure all interior locations of hospitals (i.e. health care, clinics, etc. and restaurants, public gathering places) are cleaned completely and on the appropriate schedule, while maintaining physical integrity.

### BRIEF SUMMARY OF THE DISCLOSURE

In an exemplary embodiment, an interior monitoring method, implemented by a server coupled to a plurality of sensors deployed throughout a location, the interior monitoring method includes receiving data from the plurality of sensors, wherein the plurality of sensors are configured to monitor environmental conditions and provide data based thereon which can be used to determine cleaning and effectiveness of the cleaning for the location; correlating the data; and determining whether the location has been cleaned based on the correlated data. The data can be temperature data, and the location is cleaned all or in part with hot water. The interior monitoring method can further include receiving pressure data from one or more of the plurality of sensors; and determining damage to the location based on the pressure data.

The interior monitoring method can further include providing an indication or notification related to whether or not the location has been cleaned. The interior monitoring method can further include determining a quality and frequency of cleaning of the location based on the data. The interior monitoring method can further include performing data analysis on the data to distinguish between cleaning and other events related to the location. The data can be sent to the server by each of the plurality of sensors based on a predetermined rise or fall of temperature. The location can include any of hospital rooms, bathrooms, restaurants, locker rooms. The plurality of sensors can be miniaturized and embedded in or attached to the various interior components at the location. The interior components can include walls, floors, ceilings, furniture, shower curtains, privacy curtains, medical devices and equipment, plumbing, tables, chairs, sofas, beds, counters, cabinets, shelving, and counters.

In another exemplary embodiment, an interior monitoring system includes a server comprising a network interface to a wireless network, a processor, and memory storing instructions that, when executed, cause the processor to: receive data from the plurality of sensors, wherein the plurality of sensors are configured to monitor environmental conditions and provide data based thereon which can be used to determine cleaning and effectiveness of the cleaning for the location; correlate the data; and determine whether the location has been cleaned based on the correlated data. The data can be temperature data, and the location is cleaned all or in part with hot water.

The memory storing instructions that, when executed, can further cause the processor to: receive pressure data from one or more of the plurality of sensors; and determine damage to the location based on the pressure data. The memory storing instructions that, when executed, can further cause the processor to provide an indication or notification related to whether or not the location has been cleaned. The memory storing instructions that, when executed, can further cause the processor to determine a quality and frequency of cleaning of the location based on the data. The memory storing instructions that, when executed, can further cause the processor to perform data analysis on the data to distinguish between cleaning and other events related to the location. The data can be sent to the server by each of the plurality of sensors based on a predetermined rise or fall of temperature. The location can include any of hospital rooms, bathrooms, restaurants, locker rooms. The plurality of sensors can be miniaturized and embedded in or attached to the various interior components at the location. The interior components can include walls, floors, ceilings, furniture, shower curtains, privacy curtains, medical devices and equipment, plumbing, tables, chairs, sofas, beds, counters, cabinets, shelving, and counters.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 is a block diagram of an interior monitoring system;

FIG. 2 is a block diagram of a server which may be used in the interior monitoring system, in other systems, or standalone;

FIG. 3 is block diagram of an exemplary implementation of the sensor;

FIG. 4 is a flow chart of an interior monitoring method, implemented by a server coupled to a plurality of sensors deployed throughout a location; and

FIGS. 5 and 6 are floor plan diagrams are illustrated for a hospital room and a locker room, as examples for the location in the interior monitoring system of FIG. 1.

### DETAILED DESCRIPTION OF THE DISCLOSURE

In various exemplary embodiments, building interior monitoring systems and methods are described for cleaning and the like. The systems and methods include various temperature and/or pressure sensors inserted/distributed throughout interior components in a building interior. The temperature and/or pressure sensors are coupled to a server (wired or wireless) and report data thereto. The building interior monitoring systems and methods include deploying tens to hundreds to thousands of sensors per location and continually monitoring data to determine whether the location has been cleaned or whether the location has been damaged, based on the temperature and/or pressure readings. This is particularly advantageous in environments such as hospital rooms, bathrooms, restaurants, locker rooms, etc., i.e., any environment where there is a risk for the spread of disease.

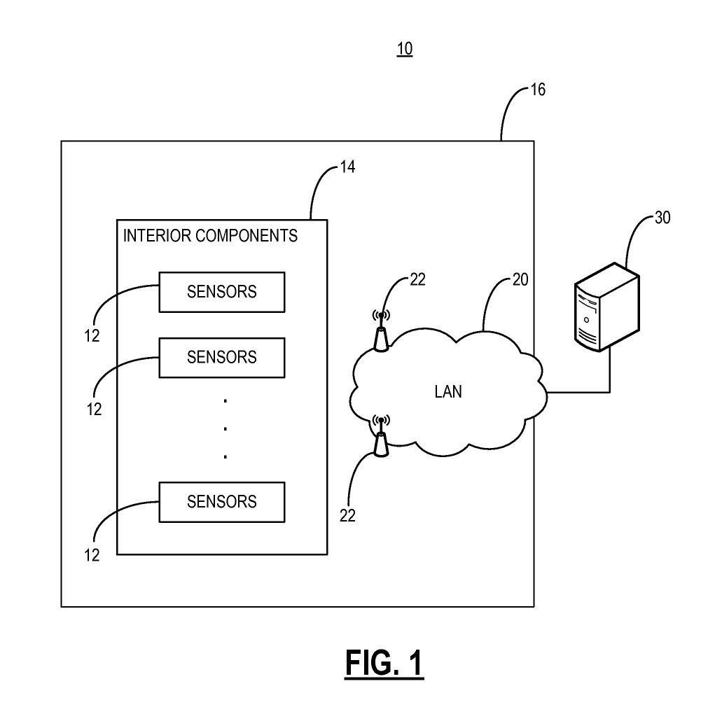

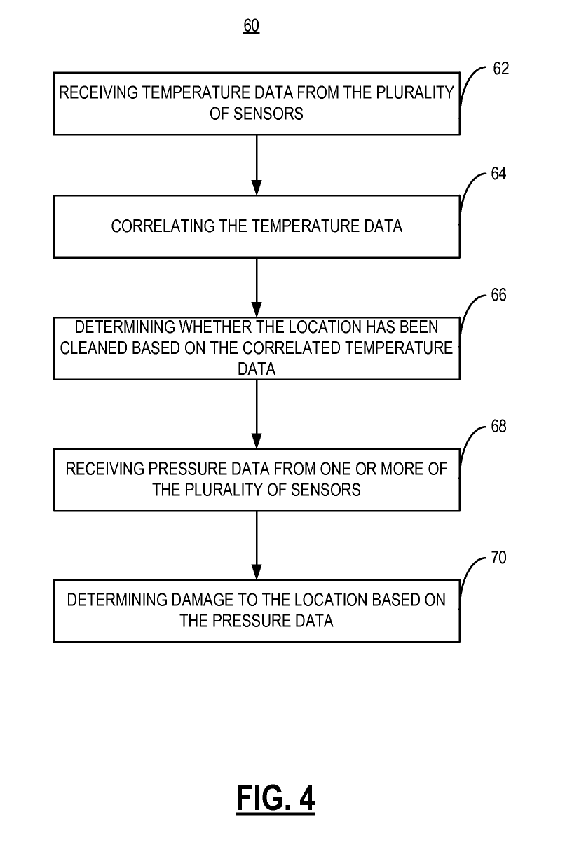

Referring to FIG. 1, in an exemplary embodiment, a block diagram illustrates an interior monitoring system 10. The interior monitoring system 10 includes a plurality of sensors 12 associated with various interior components 14 at a location 16. The sensors 12 can include temperature sensors, pressure sensors, combination temperature and pressure sensors, or any other type of sensor that can measure data which can be correlated to cleaning effectiveness, such as, for example, biosensors to measure bacteria, alcohol sensors to monitor for cleaning solution, etc. For descriptions here, the sensors 12 are referenced as temperature and/or pressure probes, but those of ordinary skill in the art will recognize that any type of sensor is contemplate so long as the data it measures can be correlated to cleaning effectiveness. An exemplary implementation of the sensors 12 is illustrated in FIG. 3.

With respect to cleaning effectiveness, one of the most common and effective methods of cleaning is hot water and/or steam. This lends itself well to using temperature sensors for the sensors 12. In addition to the hot water and/or steam, various other detergents, scouring agents, glass cleaners, etc. can be used. To disinfect, two top agents (i.e. oxidizers) are hydrogen peroxide and ozone (made by flashing ultraviolet light on surface or by putting ozone in cold water). The way forward is for hospitals and other environments to be green, and thus use less chemicals (i.e. acids/bases, alcohols, etc.) and use natural techniques, like steam, hot water and hydrogen peroxide. Thus, the sensors 12 can also measure an oxidizer. Also, the sensors 12, as pressure sensors, can measure whether a surface has been wiped or not.

The sensors 12 are miniaturized and embedded in or attached to the various interior components 14 at the location. The interior components 14 can include, without limitation, walls, floors, ceilings, furniture, curtains (shower, privacy, etc.), medical devices and equipment (hospital beds, wheelchairs, exam tables, etc.), plumbing (sinks, toilets, showers, etc.), tables, chairs, sofas, beds, counters, cabinets, shelving, counters, and the like. That is, the interior components 14 can be anything in the location 16. The interior monitoring system 10 contemplates numerous of the sensors 12 deployed throughout the location 16 to provide good coverage. Note, the sensors 12 can be associated with surfaces and components that need to be cleaned or that may be damaged as well as with surfaces and components that do not necessarily come into contact with people (e.g., ceilings, etc.). The goal of the sensor 12 deployment in the location 16 is to provide adequate coverage to provide real-time readings related to temperature and/or pressure for building interior monitoring.

The location 16 can include, without limitation, hospital rooms, medical offices, bathrooms, restaurants, locker rooms, hotel rooms, retail locations, etc. The location 16 can include a Local Area Network (LAN) 20 with one or more Access Points (APs) 22 for wireless connectivity and various wired components for wired connectivity. The sensors 12 are configured to communicate with a server 30 via the LAN 20. The APs 22 can be any wireless networking technique such as, without limitation, Bluetooth, Bluetooth Low Energy (BLE), IEEE 802.11 and variants thereof, IEEE 802.15 and variants thereof, Wireless Personal Area Network (WPAN) techniques, etc. In an exemplary embodiment, the sensors 12 at the location 16 are configured in a Wireless Sensor Network (WSN) or combination WSN and Wired Sensor Network. In an exemplary embodiment, the sensors 12 preferably be wired, such as with twisted pair connections, where possible, such as in walls, fixtures, medical equipment, etc. because the wired configuration does not require batteries. The sensors 12 are wireless where needed, such as in curtains or any other structure where it is not feasible to include wiring.

The server 30 is configured to receive data from the sensors 12 at the location and perform data analytics on the data from the sensors 12. Specifically, the server 30 is configured to detect cleaning of the location 16 based on temperature data from the sensors 12. For example, when the temperature rises, above a certain preset threshold, each of the sensors 12 can report to the server 30, which can correlate the temperature data to determine when and if the location 16 is cleaned. Specifically, the temperature data from the sensors 12 disposed throughout a room or the location 16 in the interior components 14 (e.g., in walls, floors, ceiling, privacy curtains, fixtures, etc.) can be correlated to provide a direct indication of a cleaning. In this manner, the temperature data from the sensors 12, aggregated and correlated by the server 30, can provide an indication of cleaning for the location 16. This can replace the conventional cleaning sheet that is placed on walls or doors that manually indicates cleaning. Further, data analytics associated with the temperature data can provide a qualitative measure of the cleaning as described herein.

Similarly, the sensors 12 can also provide pressure data and report back to the service 30 when the pressure rises abruptly, to indicate damage as well as cleaning. In an exemplary embodiment, the sensors 12 can be a combination of pressure and temperature sensors. In another exemplary embodiment, the sensors 12 can only be temperature sensors. With the server 20, a profile is kept, over a time interval, of what is cleaned and not, and with data analytics, other factors can be determined or surmised from the temperature and/or pressure data such as, for a hospital or medical office, a new patient in a room and the room must be cleaned before they enter, etc. Also, when a pressure sensor goes high, a team can go to inspect the damage (e.g., from a wheel chair) and then they fix the physical problem.

In a typical room or location 14, the sensors 12 are installed in the walls, floors, furniture, etc. in such places as to show the room was cleaned—which means that if the room is cleaned after all or most of the sensors 12 send a signal. From time to time, more of the sensors 12 can be added or moved to ensure that the cleaning personnel do not know where the sensors are located. The interior monitoring system 10 contemplates tens to hundreds to thousands of the sensors 12 for each of the locations 16, depending on various factors like size of the location, cleaning needs, etc.

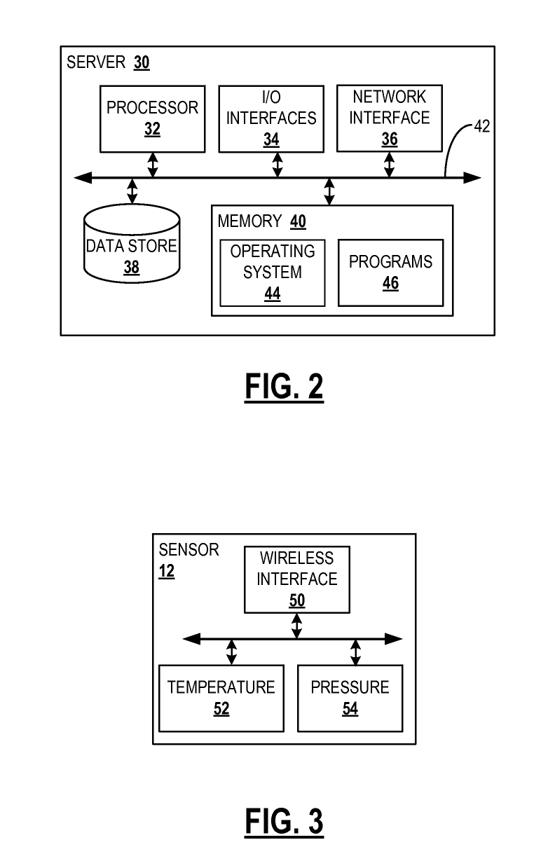

Referring to FIG. 2, in an exemplary embodiment, a block diagram illustrates a server 30 which may be used in the interior monitoring system 10, in other systems, or standalone. The server 30 may be a digital computer that, in terms of hardware architecture, generally includes a processor 32, input/output (I/O) interfaces 34, a network interface 36, a data store 38, and memory 40. It should be appreciated by those of ordinary skill in the art that FIG. 2 depicts the server 30 in an oversimplified manner, and a practical embodiment may include additional components and suitably configured processing logic to support known or conventional operating features that are not described in detail herein. The components (32, 34, 36, 38, and 40) are communicatively coupled via a local interface 42. The local interface 42 may be, for example but not limited to, one or more buses or other wired or wireless connections, as is known in the art. The local interface 42 may have additional elements, which are omitted for simplicity, such as controllers, buffers (caches), drivers, repeaters, and receivers, among many others, to enable communications. Further, the local interface 42 may include address, control, and/or data connections to enable appropriate communications among the aforementioned components.

The processor 32 is a hardware device for executing software instructions. The processor 32 may be any custom made or commercially available processor, a central processing unit (CPU), an auxiliary processor among several processors associated with the server 30, a semiconductor-based microprocessor (in the form of a microchip or chip set), or generally any device for executing software instructions. When the server 30 is in operation, the processor 32 is configured to execute software stored within the memory 40, to communicate data to and from the memory 40, and to generally control operations of the server 30 pursuant to the software instructions. The I/O interfaces 34 may be used to receive user input from and/or for providing system output to one or more devices or components. User input may be provided via, for example, a keyboard, touch pad, and/or a mouse. System output may be provided via a display device and a printer (not shown). I/O interfaces 34 may include, for example, a serial port, a parallel port, a small computer system interface (SCSI), a serial ATA (SATA), a fibre channel, Infiniband, iSCSI, a PCI Express interface (PCI-x), an infrared (IR) interface, a radio frequency (RF) interface, and/or a universal serial bus (USB) interface.

The network interface 36 may be used to enable the server 30 to communicate on a network, such as the Internet, the WAN 101, the enterprise 200, and the like, etc. The network interface 36 may include, for example, an Ethernet card or adapter (e.g., 10BaseT, Fast Ethernet, Gigabit Ethernet, 10 GbE) or a wireless local area network (WLAN) card or adapter (e.g., 802.11a/b/g/n). The network interface 36 may include address, control, and/or data connections to enable appropriate communications on the network. A data store 38 may be used to store data. The data store 38 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 38 may incorporate electronic, magnetic, optical, and/or other types of storage media. In one example, the data store 38 may be located internal to the server 30 such as, for example, an internal hard drive connected to the local interface 42 in the server 30. Additionally in another embodiment, the data store 38 may be located external to the server 30 such as, for example, an external hard drive connected to the I/O interfaces 34 (e.g., SCSI or USB connection). In a further embodiment, the data store 38 may be connected to the server 30 through a network, such as, for example, a network attached file server.

The memory 40 may include any of volatile memory elements (e.g., random access memory (RAM, such as DRAM, SRAM, SDRAM, etc.)), nonvolatile memory elements (e.g., ROM, hard drive, tape, CDROM, etc.), and combinations thereof. Moreover, the memory 40 may incorporate electronic, magnetic, optical, and/or other types of storage media. Note that the memory 40 may have a distributed architecture, where various components are situated remotely from one another, but can be accessed by the processor 32. The software in memory 40 may include one or more software programs, each of which includes an ordered listing of executable instructions for implementing logical functions. The software in the memory 40 includes a suitable operating system (O/S) 44 and one or more programs 46. The operating system 44 essentially controls the execution of other computer programs, such as the one or more programs 46, and provides scheduling, input-output control, file and data management, memory management, and communication control and related services. The one or more programs 46 may be configured to implement the various processes, algorithms, methods, techniques, etc. described herein.

Referring to FIG. 3, in an exemplary embodiment, a block diagram illustrates an exemplary implementation of the sensor 12. It is expected the sensor 12 is a small form-factor with low cost, such that numerous of the sensors 12 can be deployed in the location 16. The sensor 12 includes a wireless interface 50, a temperature probe 52, and/or a pressure probe 54. The wireless interface 50 allows the sensor 12 to communicate on the LAN 20 to the APs 22 and can include, without limitation, Bluetooth, Bluetooth Low Energy (BLE), IEEE 802.11 and variants thereof, IEEE 802.15 and variants thereof, Wireless Personal Area Network (WPAN) techniques, etc. The temperature probe 52 is configured to measure the temperature at or near the sensor 12, and the pressure probe 54 is configured to measure the pressure at or near the sensor 12. The sensor 12 may also include software or firmware to implement a set of instructions as described herein.

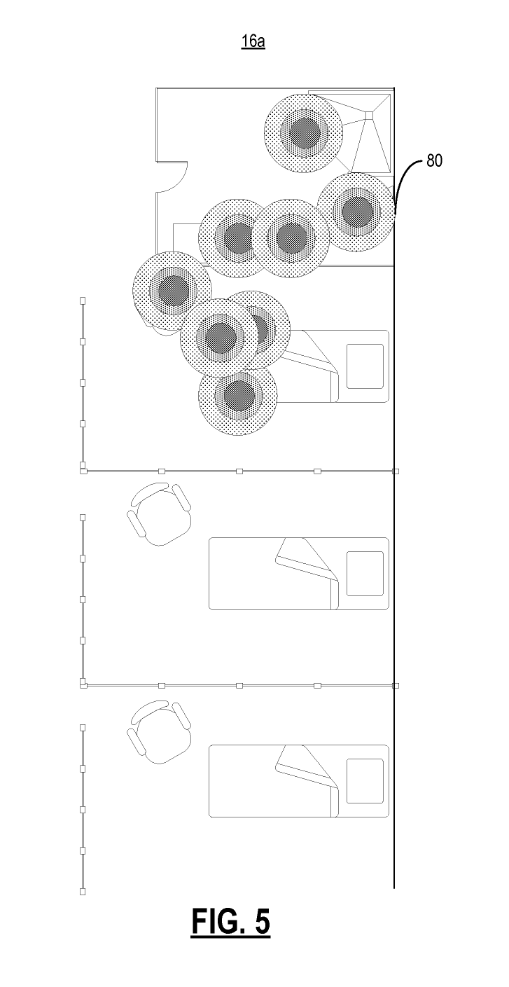

Referring to FIG. 4, in an exemplary embodiment, a flow chart illustrates an interior monitoring method 60, implemented by a server coupled to a plurality of sensors deployed throughout a location. The interior monitoring method 60 includes receiving temperature data from the plurality of sensors (step 62); correlating the temperature data (step 64); and determining whether the location has been cleaned based on the correlated temperature data (step 66). The interior monitoring method 60 can also include receiving pressure data from one or more of the plurality of sensors (step 68); and determining damage to the location based on the pressure data (step 70).

The interior monitoring method 60 can include providing an indication or notification related to whether or not the location has been cleaned. Here, the server 30 can provide a notification to a site administrator or the like, such as via a mobile device, push notification, text message, email, alert, etc. The interior monitoring method 60 can include determining a quality and frequency of cleaning of the location based on the temperature data. Here, the interior monitoring method 60 can use data analysis and historical records to determine what happens relative to a good cleaning and a poor cleaning One example may include a length of time the temperature is elevated, which can correlate to how much time is spent in a specific area of the location cleaning.

The interior monitoring method 60 can include performing data analysis on the temperature data to distinguish between cleaning and other events related to the location. The temperature data can be sent to the server by each of the plurality of sensors based on a predetermined rise or fall of temperature. The location can include any of hospital rooms, bathrooms, restaurants, locker rooms. The plurality of sensors can be miniaturized and embedded in or attached to the various interior components at the location. The interior components can include walls, floors, ceilings, furniture, shower curtains, privacy curtains, medical devices and equipment, plumbing, tables, chairs, sofas, beds, counters, cabinets, shelving, and counters.

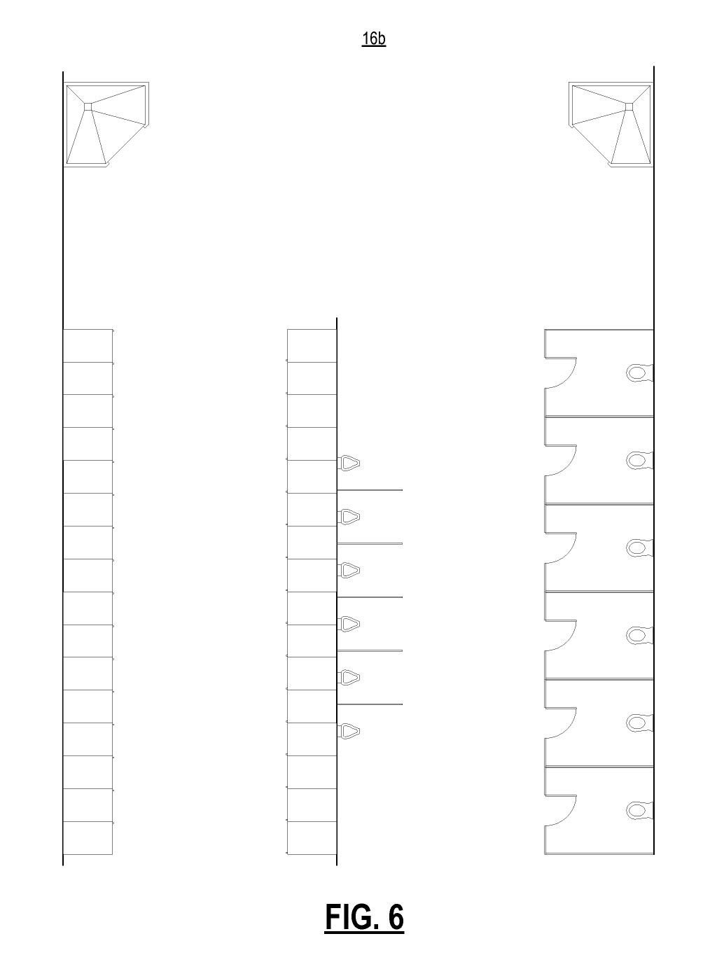

Referring to FIGS. 5 and 6, in an exemplary embodiment, floor plan diagrams are illustrated for a hospital room 16a and a locker room 16b, as examples for the location 16 in the interior monitoring system 10. In the hospital room 16a, the sensors 12 can be deployed in the bed, on the floor, on the privacy curtains, etc. In the locker room 16b, the sensors 12 can be deployed in the bed, on the floor, in the showers, etc. Various other applications are also contemplated.

For example, the sensors 12 can be visually displayed as heat maps 80 showing activity and the like. The heat maps 80 can be used to judge cleaning effectiveness. For example, assume there are 100 sensors 12 in a room, and only 75 of the sensors 12 are “lit up” showing an effective cleaning. From this data, a specific determination can be made of what has not been cleaned in the room. Taken over time and in various rooms, this data can yield patterns which can be acted upon to improve the cleaning effectiveness. For example, what are the most often missed areas in a room, etc.

It will be appreciated that some exemplary embodiments described herein may include one or more generic or specialized processors (“one or more processors”) such as microprocessors, digital signal processors, customized processors, and field programmable gate arrays (FPGAs) and unique stored program instructions (including both software and firmware) that control the one or more processors to implement, in conjunction with certain non-processor circuits, some, most, or all of the functions of the methods and/or systems described herein. Alternatively, some or all functions may be implemented by a state machine that has no stored program instructions, or in one or more application specific integrated circuits (ASICs), in which each function or some combinations of certain of the functions are implemented as custom logic. Of course, a combination of the aforementioned approaches may be used. Moreover, some exemplary embodiments may be implemented as a non-transitory computer-readable storage medium having computer readable code stored thereon for programming a computer, server, appliance, device, etc. each of which may include a processor to perform methods as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), Flash memory, and the like. When stored in the non-transitory computer readable medium, software can include instructions executable by a processor that, in response to such execution, cause a processor or any other circuitry to perform a set of operations, steps, methods, processes, algorithms, etc.

Although the present disclosure has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present disclosure, are contemplated thereby, and are intended to be covered by the following claims.

What is claimed is:

1. An interior monitoring method, implemented by a server coupled to a plurality of sensors deployed throughout a location, the interior monitoring method comprising:
receiving data from the plurality of sensors, wherein the plurality of sensors are configured to monitor environmental conditions and provide data based thereon which can be used to determine cleaning and effectiveness of the cleaning for the location;
correlating the data; and
determining whether the location has been cleaned based on the correlated data.

2. The interior monitoring method of claim 1, wherein the data is temperature data, and the location is cleaned all or in part with hot water.

3. The interior monitoring method of claim 1, further comprising:
receiving pressure data from one or more of the plurality of sensors; and
determining damage to the location based on the pressure data.

4. The interior monitoring method of claim 1, further comprising:
providing an indication or notification related to whether or not the location has been cleaned.

5. The interior monitoring method of claim 1, further comprising:
determining a quality and frequency of cleaning of the location based on the data.

6. The interior monitoring method of claim 1, further comprising:
performing data analysis on the data to distinguish between cleaning and other events related to the location.

7. The interior monitoring method of claim 1, wherein the data is sent to the server by each of the plurality of sensors based on a predetermined rise or fall of temperature.

8. The interior monitoring method of claim 1, wherein the location comprises any of hospital rooms, bathrooms, restaurants, locker rooms.

9. The interior monitoring method of claim 1, wherein the plurality of sensors are miniaturized and embedded in or attached to the various interior components at the location.

10. The interior monitoring method of claim 9, wherein the interior components comprise walls, floors, ceilings, furniture, shower curtains, privacy curtains, medical devices and equipment, plumbing, tables, chairs, sofas, beds, counters, cabinets, shelving, and counters.

11. An interior monitoring system, comprising:
a server comprising a network interface to a wireless network, a processor, and memory storing instructions that, when executed, cause the processor to:
receive data from the plurality of sensors, wherein the plurality of sensors are configured to monitor environmental conditions and provide data based thereon which can be used to determine cleaning and effectiveness of the cleaning for the location;
correlate the data; and
determine whether the location has been cleaned based on the correlated data.

12. The interior monitoring system of claim 11, wherein the data is temperature data, and the location is cleaned all or in part with hot water.

13. The interior monitoring system of claim 11, wherein the memory storing instructions that, when executed, further cause the processor to:
receive pressure data from one or more of the plurality of sensors; and
determine damage to the location based on the pressure data.

14. The interior monitoring system of claim 11, wherein the memory storing instructions that, when executed, further cause the processor to:
provide an indication or notification related to whether or not the location has been cleaned.

15. The interior monitoring system of claim 11, wherein the memory storing instructions that, when executed, further cause the processor to:
determine a quality and frequency of cleaning of the location based on the data.

16. The interior monitoring system of claim 11, wherein the memory storing instructions that, when executed, further cause the processor to:
perform data analysis on the data to distinguish between cleaning and other events related to the location.

17. The interior monitoring system of claim 11, wherein the data is sent to the server by each of the plurality of sensors based on a predetermined rise or fall of temperature.

18. The interior monitoring system of claim 11, wherein the location comprises any of hospital rooms, bathrooms, restaurants, locker rooms.

19. The interior monitoring system of claim 11, wherein the plurality of sensors are miniaturized and embedded in or attached to the various interior components at the location.

20. The interior monitoring system of claim 19, wherein the interior components comprise walls, floors, ceilings, furniture, shower curtains, privacy curtains, medical devices and equipment, plumbing, tables, chairs, sofas, beds, counters, cabinets, shelving, and counters.
